(19) United States
(12) Patent Application Publication
(10) Pub. No.: US 2021/0177806 A1
(43) Pub. Date: Jun. 17, 2021

(54) COMBINATION PHARMACEUTICAL, PROPHYLACTIC OR SUPPRESSIVE AGENT FOR DEVELOPMENT OF RESISTANCE TO PYRIMIDINE ANTIMETABOLITE, AND METHOD OF TREATING DISEASE

(71) Applicant: FUJIFILM Corporation, Tokyo (JP)

(72) Inventors: Kensuke KOMATSU, Ashigarakami-gun (JP); Hiroyuki IWAMURA, Ashigarakami-gun (JP); Chieko KINOUCHI, Ashigarakami-gun (JP)

(73) Assignee: FUJIFILM Corporation, Tokyo (JP)

(21) Appl. No.: 17/188,129

(22) Filed: Mar. 1, 2021

Related U.S. Application Data
(63) Continuation of application No. PCT/JP2019/013993, filed on Mar. 29, 2019.

(30) Foreign Application Priority Data
Sep. 3, 2018 (JP) 2018-164478

(51) Int. Cl. A61K 31/4164; A61K 31/7068; A61K 31/706; A61P 35/02

(52) U.S. Cl. CPC A61K 31/4164; A61P 35/02; A61K 31/706; A61K 31/7068

(57) ABSTRACT

An object of the present invention is to provide a combination pharmaceutical which is capable of preventing or suppressing the development of resistance to the pyrimidine antimetabolite, a prophylactic or suppressive agent for the development of resistance to a pyrimidine antimetabolite, and a method of treating a disease using the above combination pharmaceutical. The present invention provides a combination pharmaceutical comprising a pyrimidine antimetabolite and 5-hydroxy-1H-imidazole-4-carboxamide, wherein the dose per one time of Compound A is 50 to 500 mg/m² and a daily dose thereof is 100 to 1,000 mg/m².

### CROSS-REFERENCE TO RELATED APPLICATIONS

This application is a Continuation of PCT International Application No. PCT/JP2019/13993 filed on Mar. 29, 2019, which claims priority under 35 U.S.C § 119(a) to Japanese Patent Application No. 2018-164478 filed on Sep. 3, 2018. Each of the above application(s) is hereby expressly incorporated by reference, in its entirety, into the present application.

### TECHNICAL FIELD

The present invention relates to a combination pharmaceutical comprising a pyrimidine antimetabolite and 5-hydroxy-1H-imidazole-4-carboxamide or a salt thereof or a hydrate thereof. The present invention also relates to a prophylactic or suppressive agent for the development of resistance to a pyrimidine antimetabolite. Furthermore, the present invention relates to a method of treating a disease using the above combination pharmaceutical.

### BACKGROUND ART

5-Hydroxy-1H-imidazole-4-carboxamide (hereinafter also referred to as Compound A) or a salt thereof or a hydrate thereof has a potent anticancer action and thus is a medically useful compound as an anticancer agent (Patent Literatures 1-8). Use of Compound A for diseases such as myelodysplastic syndrome (MDS), chronic myeloid leukemia (CML) and acute myeloid leukemia (AML) has been considered (Non Patent Literatures 1-2).

On the other hand, pyrimidine antimetabolites such as azacitidine inhibits the function of RNA and DNA to interfere with the growth and division of tumor cells and normalize abnormal gene function, and thus are used for cancer treatment.

It is reported that Compound A displays cell growth inhibitory activity without cross resistance to azacitidine-resistant leukemia cell lines (Non Patent Literature 3). It is further reported that AML cell lines are treated with a combination of Compound A and azacitidine (Non Patent Literature 4).

### PATENT LITERATURES

Patent Literature 1: JP Patent Publication (Kokai) No. 53-32124 (1978)
Patent Literature 2: International Publication No. WO2009/035168
Patent Literature 3: International Publication No. WO2013/047758
Patent Literature 4: International Publication No. WO2014/112529
Patent Literature 5: International Publication No. WO2014/112530
Patent Literature 6: International Publication No. WO2014/112531
Patent Literature 7: International Publication No. WO2015/105174
Patent Literature 8: International Publication No. WO2018/051971

### NON PATENT LITERATURES

Non Patent Literature 1: Japanese Journal of Cancer and Chemotherapy Vol. 16, No. 1, p. 123-130, 1989
Non Patent Literature 2: Cancer research Vol. 42, p. 1098-1102, 1982
Non Patent Literature 3: Pharma Res Per, 4(1), 2016, e00206
Non Patent Literature 4: Leukemia Research 59 (2017) 85-92

### SUMMARY OF INVENTION

### Object to be Solved by the Invention

When pyrimidine antimetabolite such as azacitidine is used for cancer treatment, the problem is a relapse due to development of resistance. Suppressing or preventing development of resistance to pyrimidine antimetabolite is desired. Although Non Patent Literature 3 shows that no cross resistant is observed for Compound A is in azacitidine-resistant leukemia cell lines, whether or not Compound A suppresses the development of resistance to azacitidine is not clear. Furthermore, also in Non Patent Literature 4, it is unclear if Compound A is capable of suppressing the development of resistance to azacitidine in cells before the development of resistance to azacitidine.

A problem to be solved by the present invention is to provide a combination pharmaceutical comprising a pyrimidine antimetabolite and 5-hydroxy-1H-imidazole-4-carboxamide or a salt thereof or a hydrate thereof, which is capable of preventing or suppressing the development of resistance to the pyrimidine antimetabolite. Another problem to be solved by the present invention is to provide a prophylactic or suppressive agent for the development of resistance to a pyrimidine antimetabolite. Still another problem to be solved by the present invention is to provide a method of treating a disease using the above combination pharmaceutical.

### Means for Solving the Object

The present inventors have conducted intensive studies to solve the above problems and as a result have found that the above problems are solved by administering a pyrimidine antimetabolite and 5-hydroxy-1H-imidazole-4-carboxamide or a salt thereof or a hydrate thereof at a pre-determined dose and pre-determined timing, and have completed the present invention.

The present invention provides the following.

(1) A combination pharmaceutical comprising:

a pyrimidine antimetabolite; and
5-hydroxy-1H-imidazole-4-carboxamide or a salt thereof or a hydrate thereof,
wherein the dose per one time of the 5-hydroxy-1H-imidazole-4-carboxamide or a salt thereof or a hydrate thereof is 50 to 500 mg/m² and a daily dose thereof is 100 to 1,000 mg/m².

(2) The combination pharmaceutical according to (1), which prevents or suppresses development of resistance to a pyrimidine antimetabolite.

(3) The combination pharmaceutical according to (1) or (2), wherein at least one of the following improvements is seen in a patient to whom the pharmaceutical is administered:

(a) the median time of AML transformation or death is 14.0 months or more;
(b) the median time of AML transformation is 21.0 months or more;
(c) the proportion of patients who have achieved red blood cell-transfusion independence from red blood cell-transfusion dependence is 48% or more; and
(d) the proportion of patients who have achieved platelet transfusion independence from platelet transfusion dependence is 45% or more.

(4) The combination pharmaceutical according to (2), wherein the development of resistance to a pyrimidine antimetabolite is prevented or suppressed for a period of 3 months or more.

(5) The combination pharmaceutical according to any one of (1) to (4), wherein the pyrimidine antimetabolite is a compound selected from a group of azacitidine, decitabine, cytarabine and gemcitabine, or a salt thereof or a hydrate thereof.

(6) The combination pharmaceutical according to any one of (1) to (5), wherein the dose per one time of the 5-hydroxy-1H-imidazole-4-carboxamide or a salt thereof or a hydrate thereof is 370 to 430 mg/m².

(7) The combination pharmaceutical according to any one of (1) to (6), wherein the pyrimidine antimetabolite is azacitidine or a salt thereof or a hydrate thereof and the daily dose of the azacitidine or a salt thereof or a hydrate thereof is 70 to 80 mg/m².

(8) The combination pharmaceutical according to any one of (1) to (7), wherein the pyrimidine antimetabolite is azacitidine or a salt thereof or a hydrate thereof, and the azacitidine or a salt thereof or a hydrate thereof is administered for 7 days or more.

(9) The combination pharmaceutical according to any one of (1) to (8), wherein the pyrimidine antimetabolite is azacitidine or a salt thereof or a hydrate thereof, and the azacitidine or a salt thereof or a hydrate thereof is administered in multiple cycles wherein one cycle consists of a 7-day administration period and a subsequent drug withdrawal period.

(10) The combination pharmaceutical according to any one of (1) to (9), wherein the 5-hydroxy-1H-imidazole-4-carboxamide or a salt thereof or a hydrate thereof is administered for 14 days to 28 days.

(11) The combination pharmaceutical according to any one of (1) to (10), wherein the 5-hydroxy-1H-imidazole-4-carboxamide or a salt thereof or a hydrate thereof is administered one day or more before or on the day of the administration of the pyrimidine antimetabolite.

(12) The combination pharmaceutical according to any one of (1) to (11), wherein the pyrimidine antimetabolite is administered as an intravenous infusion and the 5-hydroxy-1H-imidazole-4-carboxamide or a salt thereof or a hydrate thereof is administered as an intravenous infusion or orally.

(13) The combination pharmaceutical according to any one of (1) to (12), which is for the treatment of blood cancer.

(14) The combination pharmaceutical according to any one of (1) to (13), which is for the treatment of myelodysplastic syndrome (MDS) or acute myeloid leukemia (AML).

(15) The combination pharmaceutical according to any one of (1) to (14), wherein the pyrimidine antimetabolite and the 5-hydroxy-1H-imidazole-4-carboxamide or a salt thereof or a hydrate thereof are provided in the same or a different pharmaceutical composition.

(16) A prophylactic or suppressive agent for the development of resistance to a pyrimidine antimetabolite, comprising 5-hydroxy-1H-imidazole-4-carboxamide or a salt thereof or a hydrate thereof.

(17) A method of treating a disease, comprising administering azacitidine or a salt thereof or a hydrate thereof and 5-hydroxy-1H-imidazole-4-carboxamide or a salt thereof or a hydrate thereof, wherein the daily dose of the azacitidine or a salt thereof or a hydrate thereof is 70 to 80 mg/m², the dose per one time of the 5-hydroxy-1H-imidazole-4-carboxamide or a salt thereof or a hydrate thereof is 50 to 500 mg/m² and the daily dose is 100 to 1,000 mg/m².

(A) Use of a pyrimidine antimetabolite and 5-hydroxy-1H-imidazole-4-carboxamide or a salt thereof or a hydrate thereof for producing a combination pharmaceutical, wherein the dose per one time of 5-hydroxy-1H-imidazole-4-carboxamide or a salt thereof or a hydrate thereof is 50 to 500 mg/m², a daily dose thereof is 100 to 1,000 mg/m², and the pyrimidine antimetabolite and the 5-hydroxy-1H-imidazole-4-carboxamide or a salt thereof or a hydrate thereof are simultaneously administered or the 5-hydroxy-1H-imidazole-4-carboxamide or a salt thereof or a hydrate thereof is administered before the administration of the pyrimidine antimetabolite.

(B) A pyrimidine antimetabolite and 5-hydroxy-1H-imidazole-4-carboxamide or a salt thereof or a hydrate thereof for use in a therapy comprising administering the pyrimidine antimetabolite and the 5-hydroxy-1H-imidazole-4-carboxamide or a salt thereof or a hydrate thereof simultaneously or administering the 5-hydroxy-1H-imidazole-4-carboxamide or a salt thereof or a hydrate thereof before the administration of the pyrimidine antimetabolite, wherein the dose per one time of 5-hydroxy-1H-imidazole-4-carboxamide or a salt thereof or a hydrate thereof is 50 to 500 mg/m² and a daily dose thereof is 100 to 1,000 mg/m².

### Advantageous Effects of Invention

The present invention can prevent or suppress the development of resistance to a pyrimidine antimetabolite.

### BRIEF DESCRIPTION OF DRAWING

FIG. 1 shows the results of a cell proliferation assay for examining the development of resistance to azacitidine of leukemia cell lines and the prophylactic or suppressive effect of Compound A.

### EMBODIMENTS FOR CARRYING OUT THE INVENTION

The present invention will be described in detail below. “%” used in the present invention means percent by mass unless otherwise specified. In the present invention, the numerical range shown with “to” represents a range inclusive of the value before and after “to” as the minimum and maximum value, respectively. Furthermore, in the present invention, when two or more substances corresponding to a component exist in a composition, the amount of the component means the total amount of the two or more substances in the composition unless otherwise specified.

“mg/m²” used in the present invention means a dose per body surface area.

Prevention means to inhibit the onset of disease, decrease the risk of the onset of disease, or delay the onset of disease.

Therapy means to improve the target disease or conditions or suppress their progress. Treatment means the prevention, therapy or the like of various diseases.

The present invention relates to a combination pharmaceutical comprising a pyrimidine antimetabolite and 5-hydroxy-1H-imidazole-4-carboxamide (compound A) or a salt thereof or a hydrate thereof, wherein the dose per one time of Compound A or a salt thereof or a hydrate thereof is 50 to 500 mg/m² and a daily dose thereof is 100 to 1,000 mg/m², and the pyrimidine antimetabolite and Compound A or a salt thereof or a hydrate thereof are simultaneously administered, or Compound A or a salt thereof or a hydrate thereof is administered before the administration of the pyrimidine antimetabolite.

For the combination pharmaceutical of the present invention, the pyrimidine antimetabolite and the 5-hydroxy-1H-imidazole-4-carboxamide or a salt thereof or a hydrate thereof may be provided in the same pharmaceutical composition or in a different pharmaceutical composition. “In the same pharmaceutical composition” means that the pyrimidine antimetabolite and Compound A or a salt thereof or a hydrate thereof are provided in such a manner that they are included in a single pharmaceutical composition.

The combination pharmaceutical of the present invention prevents or suppresses the development of resistance to a pyrimidine antimetabolite. A possible mechanism of prevention or suppression of the development of resistance to a pyrimidine antimetabolite of Compound A or a salt thereof or a hydrate thereof will be described below. Aza represents azacitidine.

Intracellular CTP (cytidine triphosphate) competes with Aza-CTP (an active metabolite of Aza) and an increased amount of CTP may reduce sensitivity to Aza. The amount of CTP in Aza-resistant leukemia cells has been demonstrated to be 5 times higher than that in Aza-sensitive cells, showing that Compound A or a salt thereof or a hydrate thereof has an inhibitory effect on CTP (an effect of inhibiting the increase of CTP). The increase in the amount of CTP was observed in response to the development of resistance to Aza after exposing leukemia cells to Aza for 4 mouths. Then, studies were conducted on the assumption that exposure to Compound A or a salt thereof or a hydrate thereof and Aza for a long time is effective for the recovery or retainment of sensitivity to Aza with the expectation that Compound A or a salt thereof or a hydrate thereof has an inhibitory effect on CTP increase.

The result is that a long-term (4 months) exposure of leukemia cells to a combination of Aza and Compound A.¾ hydrate caused a change in Aza sensitivity. A 4-month exposure of naive leukemia cells (SKM-1) to Aza and Compound A (1 mon or 10 μmon) resulted in almost complete elimination of the development of resistance to Aza. A 4-month exposure to only Aza caused an increase in the amount of intracellular CTP, resulting in the development of resistance. It has been shown that when Aza and Compound A were combined from the start of the treatment, the amount of intracellular CTP was kept at a level similar to that in untreated cells. These results show that Compound A or a salt thereof or a hydrate thereof prevents an increase in the amount of intracellular CTP in the process of developing resistance to Aza and thus suppresses the development of the resistance. Thus, treating an untreated MDS patient with a combination of Aza and Compound A or a salt thereof or a hydrate thereof is considered to be effective for maintaining their sensitivity to Aza.

### [1] Pyrimidine Antimetabolite

Pyrimidine base is a component of DNA which is necessary for cell proliferation. Pyrimidine antimetabolites have a structure similar to that of pyrimidine base and exhibit cell proliferation inhibitory effect when incorporated into DNA strands instead of the pyrimidine base in the step of synthesis of DNA.

Pyrimidine antimetabolites are known to be therapeutically effective when converted to an active form, i.e., a triphosphate form, in a cell, and competing with CTP. Then, it is highly likely that the mechanism of the development of resistance to pyrimidine antimetabolites other than azacitidine includes an increase in the level of intracellular CTP as is the case with azacitidine. Therefore, Compound A or a salt thereof or a hydrate thereof is considered to be capable of suppressing the development of resistance to pyrimidine antimetabolites other than azacitidine by the same mechanism as for azacitidine.

Examples of pyrimidine antimetabolites include, but are not limited to, the following compounds or a salt thereof or a hydrate thereof.
Azacitidine (also referred to as 5-azacytidine) (4-amino-1-β-D-ribofuranosyl-1,3,5-triazin-2(1H)-one)
Decitabine (5-aza-2′-deoxycytidine)
Cytarabine (arabinosylcytosine)
Gemcitabine (2′-deoxy-2′,2′-difluorocytidine)
Zebularine (2(1H)-pyrimidine-riboside)
Emtriva (2′,3′-dideoxy-5-fluoro-3′-thiacytidine)
Capecitabine (N⁴-pentyloxycarbonyl-5′-deoxy-5-fluorocytidine)
2′-cyclocytidine,
Arabinofuranosyl (arabinofuranosyl)-5-azacytidine
Dihydro-5-azacytidine
N⁴-octadecyl-cytarabine
Elaidic acid cytarabine, and
Cytosine 1-β-D-arabinofuranoside

A compound selected from the group consisting of azacitidine, decitabine, cytarabine and gemcitabine, or a salt thereof or a hydrate thereof is preferably used as a pyrimidine antimetabolite. Azacitidine or a salt thereof or a hydrate thereof is particularly preferably used.

One compound or two or more compounds may be used as a pyrimidine antimetabolite.

Examples of salts of pyrimidine antimetabolite include known salts via a basic group or acidic group.

Examples of salts of a basic group include salts with a mineral acid such as hydrochloric acid, hydrogen bromide, phosphoric acid and sulfuric acid; salts with an organic carboxylic acid such as tartaric acid, formic acid, acetic acid, fumaric acid, maleic acid, citric acid, trichloroacetic acid and trifluoroacetic acid; and salts with a sulfonic acid such as methanesulfonic acid, benzenesulfonic acid, p-toluenesulfonic acid, mesitylenesulfonic acid and naphthalenesulfonic acid.

Examples of salts of an acidic group include salts with an alkali metal such as sodium and potassium; salts with an alkaline earth metal such as calcium and magnesium; ammonium salts; and salts with a nitrogen-containing organic base such as trimethylamine, triethylamine, tributylamine, trometamol, pyridine, N,N-dimethylaniline, N-methylpiperidine, N-methylmorpholine, diethylamine, dicyclohexylamine, procaine, dibenzylamine, N-benzyl-β-phenethylamine and N,N′-dibenzylethylenediamine.

Of the above salts, pharmacologically acceptable salts are preferred.

When the pyrimidine antimetabolite is azacitidine or a salt thereof or a hydrate thereof, the daily dose of azacitidine or a salt thereof or a hydrate thereof is preferably 20 to 200 mg/m², more preferably 50 to 100 mg/m², still more preferably 60 to 90 mg/m², further preferably 70 to 80 mg/m², and particularly preferably 75 mg/m².

When the pyrimidine antimetabolite is azacitidine or a salt thereof or a hydrate thereof, it is preferable that azacitidine or a salt thereof or a hydrate thereof be administered for 7 days.

When the pyrimidine antimetabolite is azacitidine or a salt thereof or a hydrate thereof, it is preferable that azacitidine or a salt thereof or a hydrate thereof be administered in multiple cycles wherein one cycle consists of a 7-day administration period and a subsequent drug withdrawal period. The drug withdrawal period may be 7 days to 28 days, 14 days to 28 days, or 18 to 24 days.

The multiple cycles mean preferably at least 3 times, at least 4 times, at least 5 times, at least 6 times, at least 7 times, at least 8 times, at least 9 times, at least 10 times, at least 12 times or at least 15 times.

When the pyrimidine antimetabolite is cytarabine or a salt thereof or a hydrate thereof, the daily dose of cytarabine or a salt thereof or a hydrate thereof is preferably 1 g/m² to 10 g/m², more preferably 2 g/m² to 6 g/m², and further preferably 3 g/m² to 5 g/m².

When the pyrimidine antimetabolite is cytarabine or a salt thereof or a hydrate thereof, it is preferable that cytarabine or a salt thereof or a hydrate thereof be administered for preferably at least 5 days, and more preferably at least 6 days.

When the pyrimidine antimetabolite is cytarabine or a salt thereof or a hydrate thereof, it is preferable that cytarabine or a salt thereof or a hydrate thereof be administered in multiple cycles wherein one cycle consists of a 5 to 6-day administration period and a subsequent drug withdrawal period. The drug withdrawal period may be 7 days to 28 days, 14 days to 28 days, or 18 to 24 days.

The multiple cycles mean preferably at least 3 times, at least 4 times, at least 5 times, at least 6 times, at least 7 times, at least 8 times, at least 9 times, at least 10 times, at least 12 times or at least 15 times.

In an example of administration in the case where the pyrimidine antimetabolite is cytarabine or a salt thereof or a hydrate thereof, cytarabine may mixed with a 5% glucose solution or physiological saline at 2 g/m² per dose to prepare 300 to 500 mL of a solution thereof, and the solution may be administered as an intravenous infusion over 3 hours every 12 hours for a maximum of consecutive 6 days.

When the pyrimidine antimetabolite is decitabine or a salt thereof or a hydrate thereof, the daily dose of decitabine or a salt thereof or a hydrate thereof is preferably 4 to 100 mg/m², more preferably 8 to 50 mg/m², further preferably 10 to 30 mg/m², and particularly preferably 15 to 25 mg/m².

When the pyrimidine antimetabolite is decitabine or a salt thereof or a hydrate thereof, it is preferable that decitabine or a salt thereof or a hydrate thereof be administered for preferably at least 4 days or at least 5 days.

When the pyrimidine antimetabolite is decitabine or a salt thereof or a hydrate thereof, it is preferable that decitabine or a salt thereof or a hydrate thereof be administered in multiple cycles wherein one cycle consists of a 4 to 6-day administration period and a subsequent drug withdrawal period. The drug withdrawal period may be 7 days to 28 days, 14 days to 28 days, or 22 to 24 days.

The multiple cycles mean preferably at least 3 times, at least 4 times, at least 5 times, at least 6 times, at least 7 times, at least 8 times, at least 9 times, at least 10 times, at least 12 times or at least 15 times.

In an example of administration in the case where the pyrimidine antimetabolite is decitabine or a salt thereof or a hydrate thereof, decitabine may be administered in multiple cycles wherein one cycle consists of a 5 consecutive day of continuous intravenous infusion of 20 mg/m² over 1 hour or more once a day and a drug withdrawal period of 23 days.

The method of administration of pyrimidine antimetabolite may be any one of oral administration and parenteral administration (e.g., subcutaneous administration, injection, infusion and administration to the rectal region). The method of administration is preferably parenteral administration, more preferably subcutaneous administration and intravenous administration, and particularly preferably intravenous infusion.

When pyrimidine antimetabolite is parenterally administered, the pyrimidine antimetabolite may be given in a pharmaceutical composition in the form of a solution or suspension. The pharmaceutical composition for parenteral administration may contain an aqueous medium, a water miscible medium, non-aqueous medium, an antimicrobial agent, a preservative, a stabilizer, a solubility improver, a tonicity agent, a buffer, an anti-oxidant, a local anesthetic, a suspension and a dispersant, a wetting agent or an emulsifier, a complexing agent, a sequestrant and a chelating agent, a cryoprotectant, a lyoprotectant and a pH adjuster.

Examples of aqueous media include, but are not limited to, water, saline, physiological saline or phosphate buffered saline (PBS), sodium chloride injection, Ringer's injection, isotonic dextrose injection, sterile water injection, and dextrose and lactated Ringer's injection. Examples of non-aqueous media include, but are not limited to, plant-derived fixed oil, castor oil, corn oil, cottonseed oil, olive oil, peanut oil, peppermint oil, safflower oil, sesame oil, soybean oil, hydrogenated plant oil, hydrogenated soybean oil, medium chain triglyceride from coconut oil and coconut seed oil. Examples of water miscible media include, but are not limited to, ethanol, 1,3-butanediol, liquid polyethylene glycol (e.g., polyethylene glycol 300 and polyethylene glycol 400), propylene glycol, glycerol, N-methyl-2-pyrrolidone, N,N-dimethylacetamide and dimethyl sulfoxide.

Examples of antimicrobial agents or preservatives include, but are not limited to, phenol, cresol, benzyl alcohol, chlorobutanol, methyl and propyl p-hydroxybenzoate, thimerosal, benzalkonium chloride (e.g., benzenthonium chloride), methyl and propyl paraben and sorbic acid. Examples of suitable tonicity agents include, but are not limited to, sodium chloride, glycerol and dextrose.

Examples of buffers include, but are not limited to, phosphate and citrate.

Examples of antioxidants include sulfite and sodium metabisulfite.

Examples of local anesthetics include, but are not limited to, procaine hydrochloride.

Examples of suspension and dispersant include, but are not limited to, carboxymethylcellulose sodium, hydroxypropylmethylcellulose and polyvinylpyrrolidone.

Examples of emulsifies include polyoxyethylene sorbitan monolaurate, polyoxyethylene sorbitan monolaurate 80 and triethanolamine oleate.

Examples of sequestrants or chelating agents include, but are not limited to, EDTA.

Examples of pH adjustors include, but are not limited to, sodium hydroxide, hydrochloric acid, citric acid, and lactic acid.

Examples of complexing agents include, but are not limited to, cyclodextrin such as α-cyclodextrin, β-cyclodextrin, hydroxypropyl-β-cyclodextrin, sulfobutylether-β-cyclodextrin and sulfobutylether-7-β-cyclodextrin.

### [2] 5-Hydroxy-1H-imidazole-4-carboxamide (Compound A)

In the present invention, the dose per one time of Compound A or a salt thereof or a hydrate thereof is 50 to 500 mg/m², preferably 370 to 430 mg/m², and more preferably 390 to 410 mg/m². To achieve a sufficient prophylactic or suppressive effect on the development of resistance to pyrimidine antimetabolite while reducing side effects, the dose per one time is preferably 100 to 350 mg/m², preferably 180 to 320 mg/m², and preferably 200 to 300 mg/m². The dose per one time in this case is preferably 200 mg/m² or 300 mg/m².

The daily dose is 100 to 1,000 mg/m². For the number of administration, administration is preferably carried out at least two times per day. The daily dose is preferably 740 to 860 mg/m², and more preferably 780 to 820 mg/m². To achieve a sufficient prophylactic or suppressive effect on the development of resistance to pyrimidine antimetabolite while reducing side effects, the daily dose is preferably 200 to 750 mg/m², preferably 360 to 840 mg/m², and preferably 400 to 600 mg/m².

The method of administration of Compound A or a salt thereof or a hydrate thereof may be any one of oral administration and parenteral administration (e.g., subcutaneous administration, injection, infusion and administration to the rectal region). The method of administration is preferably subcutaneous administration or oral administration, and more preferably oral administration.

The period of administration of Compound A or a salt thereof or a hydrate thereof is preferably 14 days to 28 days per cycle, and more preferably 14 days to 21 days per cycle, for example, when one cycle is 28 days.

In the present invention, pyrimidine antimetabolite and Compound A or a salt thereof or a hydrate thereof are simultaneously administered, or Compound A or a salt thereof or a hydrate thereof is administered before the administration of the pyrimidine antimetabolite. Preferably, Compound A or a salt thereof or a hydrate thereof is administered one day or more before the administration of the pyrimidine antimetabolite.

Examples of salts of Compound A include known salts via a basic group or acidic group.

Examples of salts of a basic group include salts with a mineral acid such as hydrochloric acid, hydrogen bromide, phosphoric acid and sulfuric acid; salts with an organic carboxylic acid such as tartaric acid, formic acid, acetic acid, fumaric acid, maleic acid, citric acid, trichloroacetic acid and trifluoroacetic acid; and salts with a sulfonic acid such as methanesulfonic acid, benzenesulfonic acid, p-toluenesulfonic acid, mesitylenesulfonic acid and naphthalenesulfonic acid.

Examples of salts of an acidic group include salts with an alkali metal such as sodium and potassium; salts with an alkaline earth metal such as calcium and magnesium; ammonium salts; and salts with a nitrogen-containing organic base such as trimethylamine, triethylamine, tributylamine, trometamol, pyridine, N,N-dimethylaniline, N-methylpiperidine, N-methylmorpholine, diethylamine, dicyclohexylamine, procaine, dibenzylamine, N-benzyl-β-phenethylamine and N,N′-dibenzylethylenediamine.

Of the above salts, pharmacologically acceptable salts are preferred as the salt of Compound A.

Examples of hydrates of Compound A or a salt thereof include hydrates of Compound A produced by the method disclosed in JP Patent Publication (Kokai) No. 58-24569 (1983), hydrates of Compound A produced by the method disclosed in International Publication No. WO2009/035168, hydrates of Compound A produced by the method disclosed in JP Patent Application No. 2017-208570 and hydrates of Compound A synthesized by the method disclosed in Examples described later.

For Compound A, the term “Compound A or a salt thereof or a hydrate thereof” means any one selected from the group consisting of Compounds A, salts of Compound A, hydrates of Compound A and hydrates of a salt of Compound A, unless otherwise specified. The term “comprising Compound A or a salt thereof or a hydrate thereof” means that at least one selected from the group consisting of Compounds A, salts of Compound A, hydrates of Compound A and hydrates of a salt of Compound A is included, unless otherwise specified.

Compound A or a salt thereof or a hydrate thereof used in the present invention may be prepared, for example, by the method described in Preparation Example 1 described later.

When Compound A or a salt thereof or a hydrate thereof is used in the combination pharmaceutical of the present invention, Compound A or a salt thereof or a hydrate thereof may be provided in the form of a pharmaceutical composition including an additive.

The form of the pharmaceutical composition containing Compound A is preferably a tablet. The content of Compound A or a salt thereof or a hydrate thereof may be 0.3 to 95%, preferably 20 to 90%, and more preferably 40 to 85% based on the mass of the tablet.

The form of tablets, the method of their production, the additives and the explanation described in International Publication No. WO2014/112530 ([0015] to [0041]) (U.S. Patent Application No. 2015/0057324) may be cited and referred to, and the disclosure is incorporated herein.

A Preferred tablet contains Compound A.¾ hydrate, silicon dioxide and crystalline cellulose. Separation of the cap of a tablet, which is called capping, can be prevented by adding crystalline cellulose.

When crystalline cellulose is added, the content of the crystalline cellulose is preferably 0.1 to 20% by mass, more preferably 1 to 15% by mass, further preferably 2 to 12% by mass, and most preferably 3 to 10% based on the mass of the tablet.

When crystalline cellulose is added, the content of Compound A.¾ hydrate is preferably 60 to 80% by mass, and more preferably 65 to 75% by mass based on the mass of the tablet.

The above tablet may also contain other additives described in International Publication No. WO 2014/112530. For preferred types and amounts of such additives, the disclosure in International Publication No. WO2014/112530 may be cited and referred to.

### [3] Prophylactic or Suppressive Agent for Development of Resistance to Pyrimidine Antimetabolite

The present invention provides a prophylactic or suppressive agent for the development of resistance to a pyrimidine antimetabolite, comprising 5-hydroxy-1H-imidazole-4-carboxamide (Compound A) or a salt thereof or a hydrate thereof. Details of the form of the prophylactic or suppressive agent for the development of resistance to a pyrimidine antimetabolite are as described in [2] above. When used as a prophylactic or suppressive agent, the agent may be administered simultaneously with pyrimidine antimetabolite or before the administration of pyrimidine antimetabolite. When used as a prophylactic or suppressive agent, the daily dose may be the same as that in the case of use in combination with pyrimidine antimetabolite described above. The dose per one time is 50 to 500 mg/m², may be 50 to 420 mg/m², 50 to 320 mg/m², or 50 to 220 mg/m². It is preferable that the agent be administered at the above dose per one time twice a day. The daily dose is thus twice the above dose per one time.

The dose per one time is preferably 200 mg/m² or 300 mg/m², at which a sufficient prophylactic and suppressive effect on the development of resistance to pyrimidine antimetabolite is obtained while reducing side effects.

### [4] Use

The use of the combination pharmaceutical of the present invention is not particularly limited. The combination pharmaceutical may be preferably used for the treatment of blood cancer, more preferably myelodysplastic syndrome (MDS) or acute myeloid leukemia (AML), and particularly preferably MDS.

Examples of blood cancers include, but are not limited to, leukemia, MDS, malignant lymphoma and multiple myeloma.

The term “leukemia” in the present invention refers to symptoms of leukemia and symptoms occurring concurrently with leukemia.

In the present invention, “leukemia” is a general term for diseases in which tumorous hematopoietic cells uncontrollably proliferate and appear in the blood. Leukemia in which tumor cells have lost their differentiation ability is called acute leukemia and one in which tumor cells still have the differentiation ability is called chronic leukemia. Furthermore, if the origin of the tumor is myeloid cells, the disease is categorized as myeloid leukemia, and if the origin is lymphoid cells, the disease is categorized as lymphoid leukemia. Thus, leukemia is roughly classified into four types: acute myelogenous leukemia: AML, chronic myelogenous leukemia: CML, acute lymphoid leukemia: ALL and chronic lymphoid leukemia: CLL. Acute promyelocytic leukemia (APL), atypical leukemia (AUL), acute myelomonocytic leukemia (AMMoL) and juvenile chronic myelogenous leukemia (JCML) are also included in this classification. Types of leukemia also include acute monocytic leukemia (AMoL), chronic monocytic leukemia (CMoL), erythroleukemia, eosinophilic leukemia, basophilic leukemia, megakaryoblastic leukemia, plasmacytic leukemia, chloroma, chronic neutrophilic leukemia, adult T-cell leukemia, lymphosarcoma cell leukemia, hairy cell leukemia and prolymphocytic leukemia.

Myelodysplastic syndrome (MDS) is a group of heterogeneous clonal hematopoietic stem cell disorders caused by dysplastic changes in at least one hematopoietic systems including myeloid, erythroid and megakaryocytic series. The above changes mean cytopenia in at least one of the three series. Patients suffering from MDS typically develop related complications such as anemia, neutropenia (infectious disease) and thrombocytopenia (bleeding). 10% to 70% of patients with MDS generally develop acute leukemia.

### [5] Method of Treating Disease and Use

The present invention provides a method of treating a disease, comprising administering azacitidine or a salt thereof or a hydrate thereof and 5-hydroxy-1H-imidazole-4-carboxamide or a salt thereof or a hydrate thereof, wherein the daily dose of the azacitidine or a salt thereof or a hydrate thereof is 70 to 80 mg/m², the dose per one time of the 5-hydroxy-1H-imidazole-4-carboxamide or a salt thereof or a hydrate thereof is 50 to 500 mg/m² and a daily dose thereof is 100 to 1,000 mg/m², and the azacitidine or a salt thereof or a hydrate thereof and the 5-hydroxy-1H-imidazole-4-carboxamide or a salt thereof or a hydrate thereof are simultaneously administered or the 5-hydroxy-1H-imidazole-4-carboxamide or a salt thereof or a hydrate thereof is administered before the administration of the azacitidine or a salt thereof or a hydrate thereof. A preferred dose per one time and a preferred daily dose are as described in [2] above.

The present invention also provides use of a pyrimidine antimetabolite and 5-hydroxy-1H-imidazole-4-carboxamide or a salt thereof or a hydrate thereof for production of a combination pharmaceutical, wherein the dose per one time of the 5-hydroxy-1H-imidazole-4-carboxamide or a salt thereof or a hydrate thereof is 50 to 500 mg/m², a daily dose thereof is 100 to 1,000 mg/m², and the pyrimidine antimetabolite and the 5-hydroxy-1H-imidazole-4-carboxamide or a salt thereof or a hydrate thereof are simultaneously administered or the 5-hydroxy-1H-imidazole-4-carboxamide or a salt thereof or a hydrate thereof is administered before the administration of the pyrimidine antimetabolite.

The present invention also provides a pyrimidine antimetabolite and 5-hydroxy-1H-imidazole-4-carboxamide or a salt thereof or a hydrate thereof for use in a therapy comprising administering the pyrimidine antimetabolite and the 5-hydroxy-1H-imidazole-4-carboxamide or a salt thereof or a hydrate thereof simultaneously or administering the 5-hydroxy-1H-imidazole-4-carboxamide or a salt thereof or a hydrate thereof before the administration of the pyrimidine antimetabolite, wherein the dose per one time of the 5-hydroxy-1H-imidazole-4-carboxamide or a salt thereof or a hydrate thereof is 50 to 500 mg/m² and a daily dose thereof is 100 to 1,000 mg/m².

The present invention will be described with reference to the following Examples, but the present invention is not limited thereto.

### EXAMPLES

The present invention will be described with reference to Reference Examples, Synthetic Examples and Examples, but the present invention is not limited thereto. “%” means “% by mass” unless otherwise specified.

The infrared absorption spectrum (hereinafter referred to as IR) was measured by an ATR method (Attenuated Total Reflection method).

The water content was measured by the Karl Fischer method.

The seed crystal used in Examples and Comparative Examples was prepared in the same manner as in International Publication No. WO2013/047758.

### Reference Example 1

13.9 g of 5-hydroxy-1H-imidazole-4-carboxamide hydrochloride dihydrate was obtained as a pale yellow crystal in the same manner as in Example 12(1) of International Publication No. WO2013/047758 using 15.0 g of 5-hydroxy-1H-imidazole-4-carboxamide hydrochloride dihydrate.

### Synthetic Example 1

10.0 g of 5-hydroxy-1H-imidazole-4-carboxamide hydrochloride dihydrate prepared by the method described in Reference Example 1 was added to and dissolved in 120 mL of 0.45 mol/L hydrochloric acid by heating at 47° C. under nitrogen atmosphere. 10 mL of a 1.5 mol/L aqueous solution of potassium dihydrogen phosphate was added dropwise to the resulting solution at 46 to 47° C. 50 mg of 5-hydroxy-1H-imidazole-4-carboxamide.¾ hydrate was added to the resulting solution as a seed crystal, and the mixture was stirred at 46 to 48° C. for 30 minutes. After precipitation of crystal was observed in the reaction mixture, 60 mL of a 1.5 mol/L aqueous solution of potassium dihydrogen phosphate was added dropwise to the solution at 47 to 48° C. The reaction mixture was cooled to 0° C. and then stirred for 1 hour, and the crystal precipitated was collected by filtration.

The resulting crystal was divided into two and one of them was air-dried to give 3.23 g of an unwashed product of 5-hydroxy-1H-imidazole-4-carboxamide.¾ hydrate as a white crystal.

The other crystal was washed successively with a mixed solution of 5.0 mL of acetone and 10.0 mL of water and with 15.0 mL of acetone, and air-dried to give 2.94 g of a washed product of 5-hydroxy-1H-imidazole-4-carboxamide.¾ hydrate as a white crystal. IR of the two products were identical.

Water content: unwashed product: 9.8%, washed product: 8.8%

IR (cm⁻¹): 3437, 1618, 1587, 1436

The ¾ hydrate of Compound A prepared by the method described in Synthetic Example 1 was used in Example 1 below.

### Example 1: Development of Resistance of Leukemia Cell Line to Azacitidine and Prophylactic Effect of ¾ Hydrate of Compound A (Hereinafter ¾ Hydrate of Compound A Will Simply be Referred to as Compound A)

SKM-1 (National Institute of Biomedical Innovation, Health and Nutrition) was used as leukemia cell line. Cells were cultured in RPMI 1640 medium (Life Technologies) containing 1% penicillin/streptomycin and 10% fetal bovine serum (FBS) in an environment of 37° C., 5% CO₂ and saturated water vapor.

To examine the prophylactic or suppressive effect of Compound A on the development of resistance to azacitidine, 4 groups were prepared: a culture to which no drug were added (control group); a culture containing only azacitidine (Aza group); a culture containing 1 mon Compound A and azacitidine (Aza+A1 group); and a culture containing 10 mon Compound A and azacitidine (Aza+A10 group). The respective cultures were subjected to long-term culture. The concentration of azacitidine was 50 nmol/L at the start and was doubled at 2-4 week intervals. When cultured for 2 weeks after the azacitidine concentration had reached 1.6 mol/L, the treated groups were subjected to an assay for cell proliferation for azacitidine.

In the cell proliferation assay, cells were first suspended in a medium so that the number of cells was 111,111 cells/mL, and 90 μL (10,000 cells) each of the resultant was seeded in a 96-well plate (Corning Incorporated) per well. Next, 10 μL of a PBS solution of azacitidine (0, 3, 10, 30, 100 or 300 μmon) was added to the plate, and the cells were cultured in an environment of 37° C., 5% CO₂ and saturated water vapor for 72 hours.

The relative number of the cancer cells after the above incubation was measured in terms of emission intensity using Cell Titer Glo (Promega Corporation) reagent by a plate reader (Envision, PerkinElmer Inc.). With the number of cells at a concentration of azacitidine of 0 μmol/L as 100% and the emission intensity of the sample to which no cells were added as 0%, the relative value of emission intensity was calculated at each azacitidine concentration. The results of each group were plotted with the azacitidine concentration (mon) on the horizontal axis and the emission intensity (relative value) on the vertical axis.

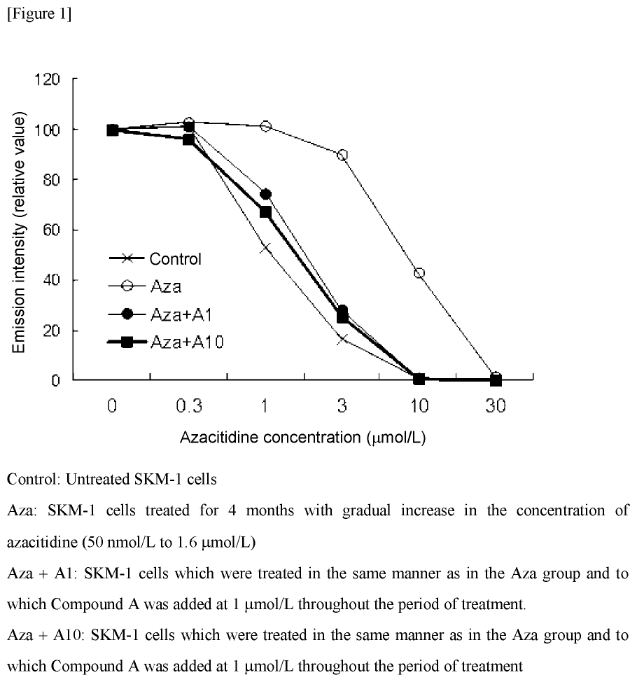

The results are shown in FIG. 1. FIG. 1 shows a graph illustrating emission intensity (relative values) of the above cancer cells. The terms in the FIGURE mean as follows. Control: Untreated SKM-1 cells

Aza: SKM-1 cells treated for 4 months with gradual increase in the concentration of azacitidine (50 nmol/L to 1.6 mol/L)
Aza+A1: SKM-1 cells which were treated in the same manner as in the Aza group and to which Compound A was added at 1 μmon throughout the period of treatment.
Aza+A10: SKM-1 cells which were treated in the same manner as in the Aza group and to which Compound A was added at 1 μmon throughout the period of treatment

As shown in FIG. 1, the emission intensity (relative value), which is considered to be an index of the number of cells, was reduced and the number of viable cells was decreased azacitidine concentration-dependently for all cancer cells. In the Aza group, the dose-response curve shifted to the right (to the side of high azacitidine concentration), and this revealed that the sensitivity to azacitidine was decreased, in other words, the resistance was developed. In contrast, the dose-response curve of Aza+A1 group and Aza+A10 group was almost the same as that of the control group and the sensitivity to azacitidine was found to be maintained. These results show that Compound A has a prophylactic or suppressive effect on the development of resistance to azacitidine. In the administration to patients, the trough level (the lowest blood concentration in a steady state) in a repeated oral administration of Compound A at 50 to 500 mg/m² twice a day is about 1 to 10 mol/L.

### Formulation Example 1

39.46 g of lactose hydrate (Pharmatose 200M available from DMV-Fonterra Excipients), 32.01 g of carmellose calcium (ECG-505 available from Nichirin Chemical Industries, Ltd.) and 20.01 g of crystalline cellulose (KG-1000 available from Asahi Kasei Corporation) were added to 276.59 g of 5-hydroxy-1H-imidazole-4-carboxamide.¾ hydrate, which is an active ingredient, to give a mixture. 12.00 g of hydroxypropyl cellulose (HPC-L available from Nippon Soda Co., Ltd.) was dissolved in 456.00 g of water. Furthermore, an aqueous solution in which 12.00 g of light anhydrous silicic acid (Aerosil-200 available from Nippon Aerosil Co., Ltd.) was dispersed was prepared. The aqueous solution prepared was sprayed on the resulting mixture, and the mixture was granulated using a fluid bed granulator (FD-MP-01 made by Powrex Corporation) and then the resultant was dried to give a granulated powder. 7.35 g of magnesium stearate (available from Merck) which had been sieved through a sieve with an opening of 180 μm was added to 360.38 g of the resulting granulated powder, and the mixture was mixed using a V-shaped mixer (Mixer Type S-5 made by Tsutsui Scientific Instruments Co., Ltd.) at 30 rpm for 60 minutes to give a mixed powder. The mixed powder obtained was compressed on a rotary tablet press (PICCOLA B-10 made by RIVA GB LTD.) using a pestle having a double R surface (12 R×3 Rmm) with a tablet diameter of 8.5 mm at a tableting pressure of 10 kN to prepare round uncoated tablets each weighing 320 mg. The amount of the active ingredient in the uncoated tablet was 200 mg. The tablet hardness of the uncoated tablet was measured by using Tablet Hardness Tester 8M (made by Schleuniger), and as a result the hardness was 76 N.

The uncoated tablet obtained was coated with a coating agent (Opadry 03A470001 TAN (hypromellose 2910: 60.00%, talc: 20.00%, titanium oxide: 18.86%, yellow ferric oxide: 1.00%, black iron oxide: 0.14%) made by Colorcon Japan LLC) in an amount of 12 mg per tablet by using a coater (DRC-200 made by Powrex Corporation) to give a round film-coated tablet. Tablets with an amount of an active ingredient of 50 mg or 100 mg were prepared in the same manner and used in Examples below.

### Example 2: Administration to MDS Patients

Compound A is administered to MDS patients (over 18 years of age) at a dose per one time of 400 mg/m² twice a day for 14 days. Administration of azacitidine is started on the day when the administration of Compound A is started. Azacitidine is administered subcutaneously or intravenously at a dose of 75 mg/m² for 7 days. The treatment cycle consists of 28 days. After the administration for 7 days, the administration is discontinued for 21 days. This Example confirms the prophylactic or suppressive effect on the development of resistance to pyrimidine antimetabolite.

### Example 3: Administration to MDS Patients

Compound A is administered to MDS patients (over 18 years of age) at a dose per one time of 400 mg/m² twice a day for 21 days. Azacitidine is administered subcutaneously or intravenously at a dose of 75 mg/m² for 7 days. The treatment cycle consists of 28 days. After the administration for 7 days, the administration is discontinued for 21 days. This Example confirms the prophylactic or suppressive effect on the development of resistance to pyrimidine antimetabolite.

### Example 4: Administration to MDS Patients

Compound A is administered to MDS patients (over 18 years of age) at a dose per one time of 300 mg/m² twice a day for 14 days. Administration of azacitidine is started on the day when the administration of Compound A is started. Azacitidine is administered subcutaneously or intravenously at a dose of 75 mg/m² for 7 days. The treatment cycle consists of 28 days. After the administration for 7 days, the administration is discontinued for 21 days. This Example confirms the prophylactic or suppressive effect on the development of resistance to pyrimidine antimetabolite. In Example 4, side effects in patients can be reduced. The day of the start of administration of Compound A may be earlier than the day of the start of administration of azacitidine. Furthermore, Compound A may be administered twice a day for 21 days as in Example 3.

### Example 5: Administration to MDS Patients

Compound A is administered to MDS patients (over 18 years of age) at a dose per one time of 200 mg/m² twice a day for 14 days. Administration of azacitidine is started on the day when the administration of Compound A is started. Azacitidine is administered subcutaneously or intravenously at a dose of 75 mg/m² for 7 days. The treatment cycle consists of 28 days. After the administration for 7 days, the administration is discontinued for 21 days. This Example confirms the prophylactic or suppressive effect on the development of resistance to pyrimidine antimetabolite. In Example 4, side effects in patients can be reduced. The day of the start of administration of Compound A may be earlier than the day of the start of administration of azacitidine. Furthermore, Compound A may be administered twice a day for 21 days as in Example 3.

For the MDS patients in Examples 2 to 5, the therapeutic effect is evaluated for the following items in comparison with the outcome of an azacitidine single agent+BSC (Best Supportive Care: Support therapy, transfusion, administration of antibiotics, analgesics, antipyretics, antiemetics, etc. as required) (International Phase III Test “Aza-001”). The period for which the development of resistance to pyrimidine antimetabolite is prevented or suppressed in the above patients is preferably 3 months or more, preferably 4 months or more, preferably 5 months or more, preferably 6 months or more, and preferably 1 year or more.

### <Evaluation of Therapeutic Effect 1>

Survival (median). The outcome with azacitidine is 24.5 months in AZA-001 test. The outcome is expected to be higher in the present invention. The survival is preferably 25 months or more, preferably 26 months or more, and preferably 28 months or more.

### <Evaluation of Therapeutic Effect 2>

2-year survival rate. The outcome with azacitidine was 50.8% in AZA-001 test. The outcome is expected to be higher in the present invention. The 2-year survival rate is preferably 51% or more, preferably 53% or more, and preferably 55% or more.

### <Evaluation of Therapeutic Effect 3>

Median Time of AML Transformation or Death

The outcome with azacitidine is 13.0 months in AZA-001 test. The outcome is expected to be higher in the present invention. The median time of AML transformation or death is preferably 14.0 months or more, preferably 15.0 months or more, preferably 17.0 months or more, and preferably 20.0 months or more.

### <Evaluation of Therapeutic Effect 4>

Median Time of AML Transformation

The outcome with azacitidine is 20.7 months in AZA-001 test. The outcome is expected to be higher in the present invention. The median time of AML transformation is preferably 21.0 months or more, preferably 22.0 months or more, preferably 24.0 months or more, and preferably 27.0 months or more.

### <Evaluation of Therapeutic Effect 5>

### Survival Rate

The outcome in the present invention is expected to be higher than the outcome with azacitidine in AZA-001 test (the Kaplan-Meier curve of survival). The 1-year survival rate is preferably 70% or more, preferably 71% or more, preferably 72% or more, preferably 75% or more, and preferably 80% or more.

### <Evaluation of Therapeutic Effect 6>

### Hematologic Response Rate According to IWG Criteria (2000 Edition)

The outcome of hematologic response rate with azacitidine was 6.7% in AZA-001 test. The outcome is expected to be higher in the present invention. The hematologic response rate is preferably 7% or more, preferably 8% or more, and preferably 9% or more.

TABLE 1
<IWG Criteria (2000 edition)>

| | | Criteria (sustained for |
|---|---|---|
| Category | Pretreatment | at least 2 months) |
| Erythroid (HI-E) | Hemoglobin level of less | Increase of more than 2 g/dL |
| | than 11 g/dL | |
| | Red blood cell (RBC)- | Independent |
| | transfusion dependence | |
| Platelet (HI-P) | Less than 100,000/mm³ | Increase of 30,000/mm³ or |
| | | more |
| | Platelet-transfusion | Platelet count stable |
| | dependence | transfusion-independently |
| | Less than 100,000/mm³ | Increase in platelet count by |
| | | 50% or more in the range of |
| | | more than 10,000/mm³ and |
| | | less than 30,000/mm³ |
| Neutrophil (HI-N) | Less than 1,500/mm³ | Larger one of the following: |
| | | increase by 100% or more or |
| | | increase of more than |
| | | 500/mm³ |
| | | Increase by 100% or more |
| | | and less than 500/mm³ |
| Progression or relapse after | | At least 50% decrement from |
| hematological improvement | | maximum response levels in |
| | | granulocytes or platelets; |
| | | Reduction in hemoglobin |
| | | level by 2 g/dL or more; or |
| | | Transfusion dependence |

### <Evaluation of Therapeutic Effect 7>

### Hematological Improvement According to IWG Criteria (2000 Edition)

The outcome of hematological improvement with azacitidine was 49.2% in AZA-001 test. The outcome is expected to be higher in the present invention.

The hematological improvement is preferably 50% or more, preferably 52% or more, and preferably 54% or more.

### <Evaluation of Therapeutic Effect 8>

### Need for Red Blood Cell Transfusion

The outcome with azacitidine in AZA-001 test is that 45.0% of patients achieved red blood cell transfusion-independence from dependence, in other words, did not receive red blood cell transfusions for consecutive 56 days or more during the test. The outcome is expected to be higher in the present invention.

The ratio of patients who have achieved red blood cell transfusion-independence from dependence is preferably 48% or more, preferably 50% or more, preferably 55% or more, and preferably 60% or more.

### <Evaluation of Therapeutic Effect: 9>

### Need for Platelet Transfusion

The outcome with azacitidine in AZA-001 test is that 42.1% of patients achieved platelet transfusion-independence from dependence, in other words, did not receive platelet transfusions for consecutive 56 days or more during the test. The outcome is expected to be higher in the present invention.

The ratio of patients who have achieved platelet transfusion-independence from dependence is preferably 45% or more, preferably 50% or more, preferably 55% or more, and preferably 60% or more.

### INDUSTRIAL APPLICABILITY

The present invention is useful as a therapeutic agent for cancer such as blood cancer.

1. A method of treating a disease, comprising administering a pyrimidine antimetabolite and 5-hydroxy-1H-imidazole-4-carboxamide or a salt thereof or a hydrate thereof, wherein the dose per one time of the 5-hydroxy-1H-imidazole-4-carboxamide or a salt thereof or a hydrate thereof is 50 to 500 mg/m² and a daily dose thereof is 100 to 1,000 mg/m².

2. The method according to claim 1, which prevents or suppresses development of resistance to a pyrimidine antimetabolite.

3. The method according to claim 1 wherein at least one of the following improvements is seen in a patient to whom the pharmaceutical is administered:
(a) the median time of AML transformation or death is 14.0 months or more;
(b) the median time of AML transformation is 21.0 months or more;
(c) the proportion of patients who have achieved red blood cell-transfusion independence from red blood cell-transfusion dependence is 48% or more; and
(d) the proportion of patients who have achieved platelet transfusion independence from platelet transfusion dependence is 45% or more.

4. The method according to claim 2, wherein the development of resistance to a pyrimidine antimetabolite is prevented or suppressed for a period of 3 months or more.

5. The method according to claim 1, wherein the pyrimidine antimetabolite is a compound selected from a group of azacitidine, decitabine, cytarabine and gemcitabine, or a salt thereof or a hydrate thereof.

6. The method according to claim 1, wherein the dose per one time of the 5-hydroxy-1H-imidazole-4-carboxamide or a salt thereof or a hydrate thereof is 370 to 430 mg/m².

7. The method according to claim 1, wherein the pyrimidine antimetabolite is azacitidine or a salt thereof or a hydrate thereof and the daily dose of the azacitidine or a salt thereof or a hydrate thereof is 70 to 80 mg/m².

8. The method according to claim 1, wherein the pyrimidine antimetabolite is azacitidine or a salt thereof or a hydrate thereof, and the azacitidine or a salt thereof or a hydrate thereof is administered for 7 days or more.

9. The method according to claim 1, wherein the pyrimidine antimetabolite is azacitidine or a salt thereof or a hydrate thereof, and the azacitidine or a salt thereof or a hydrate thereof is administered in multiple cycles wherein one cycle consists of a 7-day administration period and a subsequent drug withdrawal period.

10. The method according to claim 1, wherein the 5-hydroxy-1H-imidazole-4-carboxamide or a salt thereof or a hydrate thereof is administered for 14 days to 28 days.

11. The method according to claim 1, wherein the 5-hydroxy-1H-imidazole-4-carboxamide or a salt thereof or a hydrate thereof is administered one day or more before or on the day of the administration of the pyrimidine antimetabolite.

12. The method according to claim 1, wherein the pyrimidine antimetabolite is administered subcutaneously or as an intravenous infusion and the 5-hydroxy-1H-imidazole-4-carboxamide or a salt thereof or a hydrate thereof is administered as an intravenous infusion or orally.

13. The method according to claim 1, which is for the treatment of blood cancer.

14. The method according to claim 1, which is for the treatment of myelodysplastic syndrome or acute myeloid leukemia.

15. The method according to claim 1, wherein the pyrimidine antimetabolite and the 5-hydroxy-1H-imidazole-4-carboxamide or a salt thereof or a hydrate thereof are provided in the same or a different pharmaceutical composition.

16. A method for prophylaxis or suppression of the development of resistance to a pyrimidine antimetabolite, comprising administering 5-hydroxy-1H-imidazole-4-carboxamide or a salt thereof or a hydrate thereof.

17. A method of treating a disease, comprising administering azacitidine or a salt thereof or a hydrate thereof and 5-hydroxy-1H-imidazole-4-carboxamide or a salt thereof or a hydrate thereof, wherein the daily dose of the azacitidine or a salt thereof or a hydrate thereof is 70 to 80 mg/m², the dose per one time of the 5-hydroxy-1H-imidazole-4-carboxamide or a salt thereof or a hydrate thereof is 50 to 500 mg/m² and a daily dose thereof is 100 to 1,000 mg/m².
